(19) United States
(12) Patent Application Publication
(10) Pub. No.: US 2004/0105907 A1
(43) Pub. Date: Jun. 3, 2004

(54) Anti-inflammatory effects of the partially purified extract of radix stephaniae tetrandrae

(75) Inventors: Chieh-Fu Chen, Taipei (TW); Cheng-Jen Chou, Taipei (TW); Yuh-Chiang Shen, Taipei (TW)

Correspondence Address: BIRCH STEWART KOLASCH & BIRCH, PO BOX 747, FALLS CHURCH, VA, 22040-0747, US

(73) Assignee: TCM Biotech International CORP., Taipei (TW)

(21) Appl. No.: 10/633,657

(22) Filed: Aug. 5, 2003

(30) Foreign Application Priority Data
Aug. 5, 2002 (TW) 0911-18048

(51) Int. Cl. A61K 35/78

(52) U.S. Cl. 424/773

(57) ABSTRACT

This invention relates to inflammatory responses in isolated peripheral human neutrophils that studied in the presence or absence of specially processed Radix Stephaniae tetrandrae (SPRST). We conclude that SPRST exerts anti-inflammatory effects by interfering with reactive oxygen species (ROS) production and calcium (Ca²⁺) influx through G-protein modulation to prevent Mac-1 up-regulation and firm adhesion by neutrophils during activation.Thns, SPRST may be clinical beneficial in the prevention of cardiovascular disease, or other diseases related to over activation of neutrophil.

### PRIORITY

This application claims the priority date of the Taiwan (R.O.C.) application entitled Anti-inflammatory effects of the partially purified extract of Radix Stephaniae tetrandrae filed on 5, Aug. 2002 with serial number0911-18048.

### BACKGROUND OF THE INVENTION

1. Field of the Invention

This invention relates to inflammatory responses in isolated peripheral human neutrophils that studied in the presence or absence of specially processed Radix Stephaniae tetrandrae (SPRST). The more particularly relates to SPRST exerts anti-inflammatory effects by interfering with reactive oxygen species (ROS) production and calcium (Ca²⁺) influx through G-protein modulation to prevent Mac-1 up-regulation and firm adhesion by neutrophils during activation.

2. Background of the Invention

The Chinese traditional medicine Radix Stephaniae tetrandrae (‘Fen-Fan-Chi’) is the dry root of Stephania tetrandra S. Moore (Menispermaceae). Major components in Radix Stephaniae tetrandrae (RST) are alkaloids that can be classified as bisbenzylisoquinoline, protoberberine, morphinane and phenanthrene types. The main active constituents in Radix Stephaniae tetrandrae are tetrandrine (Tet), fangchinoline (Fan), oblongine (Obl), cyclanoline (Cyc), menisine and menisidine. Another plant used instead of Stephania tetrandra S. Moore is Radix Cocculus trilobus (Menispermace) in which contains trilobine, isotrilobine, magnoflorine, trilobamine, coclobine, menisarine and normenisarine. Two other alternative medicines originated from the family of Aristolochiaceae named as ‘Radix Aristolochia weslandi’ and ‘Radix Aristolochia heterophylla’ displaying similar appearances as Radix Stephaniae tetrandrae or Radix Cocculus trilobus are notorious for their nephrotoxicity by their toxic component ‘aristolochic acid’.

### Description of the oth Prior Art.

Radix Stephania tetrandrae, dry roots of Stephaniae tetrandrine S. Moore (Menispermaceae), is officially and traditionally used as an analgesic and anti-hypertension drug in China. The main chemical constituents in Radix Stephania tetrandrae are tetrandrine (Tet) and fangchinoline (Fan) (Tang, W. and Eisenbrand, G., Chinese Drugs of Plant Origin, 963-978, 1992). Tet is the best characterized as calcium-entry blocker (Felix, J. P. et al., Biochemistry, 31, 11793-11800,1992); it exhibits numerous pharmacological activities including modulating cardiovascular disorders (Huang, Y.-T. and Hong, C.-Y, Cardiovasc Drug Rev, 16, 1-15, 1998), anti-tumor (DeConti, R. C. et al., Cancer Res Am Soc Clin Onco, 16, 96, 1975) as well as anti-inflammatory effects (Shen,Y.-C. et al., Mol Pharmacol, 55, 186-193, 1999). Fan had been shown to be less potent than Tet as a vasodilator and calcium channel blocker (Kim, H. S. et al., J Ethnopharmacol, 58, 117-123, 1997). Fan also exhibits antioxidant (Ma, J. Y. et al., Exp Lung Res, 18, 829-843, 1992), anti-inflammatory effects in the mouse ear edema model (Choi, H. S. et al., J Ethnopharmacol, 69, 173-179, 2000) and proinflammatory cytokines released by human peripheral monocyte (Onai, N. et al., Planta Medica, 61, 497-501, 1995).

We have demonstrated that the partially purified extract of S. tetrandrae containing around 10% Tet produces equipotent cardioprotective effect as Tet on the isolated ischaemia/reperfused (I/R) rat heart but circumventing the side effects of verapamil (Yu, X.-C. et al., Life Science, 68, 2863-2872, 2001). However, the mechanism (s) of action have remained unclear. It is well known that activation and transmigration of neutrophils to infarct myocardium plays a crucial role in the myocardial I/R injury (Williams, F. M., Role of neutrophils in reperfusion injury, In: Immunopharmacology of Neutrophils, 245-257, 1994) and neutrophil infiltration has been emphasized to be an essential pathological factor contributing to the induction of myocardial I/R injury (Engler, R. L. et al., Am J Physiol 251: H93-100, 1986). Infiltration of neutrophils into tissue injury begins with the binding of neutrophils to the endothelium, followed by their extravasation into tissues (Albelda, S. M. et al., FASEB J., 8, 504-512, 1994).

This physiology comprises distinct phases including rolling, activation, firm adhesion and transmigration (Ley, K., Cardiovasc Res, 32, 733-742, 1996). A molecular explanation for these phases involves specific interactions of various cell adhesion molecules expressed on neutrophil and endothelium. These fall into three major superfamilies: (1) the selectins and their mucin ligands, (2) the integrins, and (3) their extracellular matrix or immmunoglobulin superfamily ligands (Brown, E., Semin Hematol 34, 319-326, 1997). While the selectins are important for rolling, firm adhesion and transmigration of neutrophils are essentially beta 2 integrin dependent (Arfors, K. E., et al., Blood 69, 338-340, 1987; Werr, J., et al., J Leukoc Biol 68, 553-560,2000). The beta 2 integrins comprise a group of heterodimeric glycoproteins with CD11b/CD18 (Mac-1) being the principal form elevated on neutrophils during myocardial I/R activation (Dreyer, W. J. et al., Circ Res 65, 1751-1762, 1989). Thus, prevention of Mac-1 mediated firm adhesion and/ or transmigration of neutrophil into site of tissue injury is a potential target for drugs to control inflammation. Besides, it has been demonstrated that reactive oxygen species (ROS) could modulate leukocyte Mac-1 expression and leukocyte endothelial adhesion, and both could be diminished by antioxidants (Serrano, C. V. J. et al., Biochim Biophys Acta 1316, 191-202, 1996). Furthermore, antagonizing calcium influx could impair Mac-1 dependent neutrophil adhesion (Perry, L. et al., Brit J Pharmacol 110, 1630-1634, 1993).

In this study we confirmed that a specially processed extract of S. tetrandrae (SPRST), containing only 1.3% of Tet and 0.7% of Fan, inhibited the neutrophil firm adhesion and transmigration. We hypothesized that interference with the upregulation of adhesion molecules may be involved in the effect. As remarked above, adhesion and transmigration of neutrophils is Mac-1 dependent and could be modulated by reactive oxygen species (ROS) and calcium mobilization. Therefore, N-formyl-methionyl-leucyl-phenylalanine (fMLP) or leukotriene B₄ (LTB₄) induced firm adhesion and transmigration as well as ROS production and calcium mobilization by neutrophils were analyzed to investigate the effects of SPRST. In particular, Mac-1 expression on the surface of neutrophils was examined.

### SUMMARY OF THE INVENTION

This invention relates to a specially processed extract of S. tetrandrae (SPRST) that exerts anti-inflammatory effects by interfering with ROS production and calcium influx through G-protein modulation to prevent Mac-1 (CD11b/CD 18)-dependent neutrophil activation and firm adhesion. This invention chiefly consists of introducing a pharmaceutical compound that has anti-inflammatory effect. The invention uses a specially processed extract of S. tetrandrae (SPRST) as the main component but various diluents and excipients could be included when necessary.

While the invention is susceptible to various modifications and alternative forms, certain illustrative embodiments thereof have been shown by way of example in the drawing and will herein be described in detail. It should be understood that it is not intended to limit the invention to the particular forms disclosed, but the intention is to cover all modifications, equivalents, and alternatives falling within the spirit and scope of the invention, as defined by the appended claims.

### BRIEF DESCRIPTION OF THE DRAWINGS

The invention will now be described by way of example with reference to the accompanying Tables and Figures in which:

FIG. 1 HPLC pattern of the active principles of SPRST including Tet, Fan, Cyc, and Obl.

FIG. 1A blank control of solvent

FIG. 1B shown HPLC pattern of four alkaloids

FIG. 1C shown HPLC pattern of four alkaloids and aristolochic acid

1. oblongine (Obl)

2. cyclanoline (Cyc)

3. fangchinoline (Fan)

4. tetrandrine (Tet )

FIG. 2 Mean concentration-response curves for SPRST, Tet, or Fan in the inhibition of fMLP-induced neutrophil firm adhesion.

Neutrophils (1×10⁷/ml) were loaded with 1 μM of BCECF-AM for 20 min at 37° C. and washed twice. BCECF-labeled neutrophils (5×10⁵/ml) were then pretreated with 1-10 μg/ml of SPRST, Tet, or Fan for 10 min at 37° C., and plated into fibrinogen-coated 24-well plate. After stimulating with 1 μM of fMLP for an additional 15 min at 37° C., non-adherent cells were washed off and adherent cells were quantified by measuring fluorescence intensity. Values are mean±S.E.M. (n=6). *P<0.05, as compared to samples receiving fMLP alone.

1. fMLP and Fan

2. fMLP and Tet

3. fMLP and SPRST

FIG. 3 Mean concentration-response curves for SPRST, Tet, or Fan in the inhibition of LTB₄-induced neutrophil firm adhesion.

Neutrophils (1×10⁷/ml) were pre-loaded with of BCECF-AM (1 μM) for 20 min at 37° C. and washed twice. BCECF-labeled neutrophils (5×10⁵/ml) were then pretreated with 1-10 μg/ml of SPRST, Tet, or Fan for 10 min at 37° C., and plated into fibrinogen-coated 24-well plate. After stimulating with 0.1 μM LTB₄ for an additional 15 min at 37° C., non-adherent cells were washed off and adherent cells were quantified by measuring fluorescence intensity. Values are mean±S.E.M. (n=6). *P<0.05, as compared to samples receiving LTB₄ alone.

1. LTB₄ and Fan

2. LTB₄ and Tet

3. LTB₄ and SPRST

FIG. 4 Mean concentration-response curves for SPRST, Tet, or Fan in the inhibition of fMLP-induced neutrophil transmigration.

Neutrophils (1×10⁷/ml) were loaded with BCECF-AM (1 μM) for 20 min at 37° C. and washed twice. BCECF-labeled neutrophils (5×10⁵/ml) were pretreated with 1-10 μg/ml of SPRST, Tet, or Fan for 10 min at 37° C., and then plated into upper chamber of fibrinogen-coated inserts. After stimulating with 1 μM of fMLP in the lower chamber for an additional 60 min at 37° C., transmigrated cells in the lower chambers were quantified by measuring fluorescence intensity. Values are mean±S.E.M. (n=6). *P<0.05, as compared to samples receiving fMLP.

1. fMLP and Fan

2. fMLP and Tet

3. fMLP and SPRST

FIG. 5 Mean concentration-response curves for SPRST, Tet, or Fan in the inhibition of LTB₄-induced neutrophil transmigration.

Neutrophils (1×10⁷/ml) were loaded with BCECF-AM (1 μM) for 20 min at 37° C. and washed twice. BCECF-labeled neutrophils (5×10⁵/ml) were pretreated with 1-10 μg/ml of SPRST, Tet, or Fan for 10 min at 37° C., and then plated onto upper chamber of fibrinogen-coated inserts. After stimulating with 0.1 μM LTB₄ in the lower chamber for an additional 60 min at 37° C., transmigrated cells in the lower chambers were quantified by measuring fluorescence intensity. Values are mean±S.E.M. (n=6). *P<0.05, as compared to samples receiving LTB₄ alone.

1. LTB₄ and Fan

2. LTB₄ and Tet

3. LTB₄ and SPRST

FIG. 6 Effects of SPRST, Tet, or Fan on fMLP-induced Mac-1 upregulation.

Flow cytometric analysis of total Mac-1 levels on the cell surface of neutrophils. Control neutrophils received neither SPRST nor fMLP treatment. SPRST (10 μg/ml)-pretreated sample, designated ‘fMLP+SPRST, were stimulated with 1 μM of fMLP.

Statistical summaries of fMLP-upregulated Mac-1 expression in the presence or absence of 5-10 μg/ml of SPRST, Tet, or Fan. Net increase in mean channel fluorescence (MCF) was calculated by subtracting the MCF value from sample receiving non-specific IgG₁ staining (70±12). The control value is 112±12. Values represent the mean±S.E.M. of MCF (n=3-5) experiments. *P<0.05, as compared to samples receiving fMLP.

1. Control (drug free)

2. fMLP only

3. fMLP and 5 μg/ml SPRST

4. fMLP and 10 μg/ml SPRST

5. fMLP and 5 μg/ml Tet

6. fMLP and 10 μg/ml Tet

7. fMLP and 5 μg/ml Fan

8. FMLP and 10 μg/ml Fan

FIG. 7 Effects of SPRST, Tet, or Fan on LTB₄-induced Mac-1 upregulation.

Flow cytometric analysis of total Mac-1 levels on the cell surface of neutrophils. Control neutrophils received neither SPRST nor LTB₄ treatment. SPRST (10 μg/ml)-pretreated sample, designated as ‘LTB₄+SPRST, were stimulated with 0.1 μM LTB₄.

Statistical summaries of LTB₄- (lower panel) upregulated Mac-1 expression in the presence or absence of 5-10 μg/ml of SPRST, Tet, or Fan. Net increase in mean channel fluorescence (ΔMCF) was calculated by subtracting the MCF value from sample receiving non-specific IgG₁ staining (70±12). The control value is 113±10. Values represent the mean±S.E.M. of AMCF (n=3-5) experiments. *P<0.05, as compared to samples receiving LTB₄ alone. Control neutrophils

1. LTB₄ only

3. LTB₄ and 5 μg/ml SPRST

4. LTB₄ and 10 μg/ml SPRST

5. LTB₄ and 5 μg/ml Tet

6. LTB₄ and 10 μg/ml Tet

7. LTB₄ and 5 μg/ml Fan

8. LTB₄ and 10 μg/ml Fan

FIG. 8 Effects of SPRST, Tet, or Fan on fMLP-induced ROS (H₂O₂) production by flow cytometry.

Neutrophils (1×10⁶/ml) were incubated at 37° C. for 5 min with DCFH-DA (20 μM). After labeling, cells were pretreated with 5-10 μg/ml SPRST or other chemicals for 10 min and stimulated with fMLP (1 μM). Production of H₂O₂ was then determined 30 min later by flow cytometry. Flow cytometric analysis of H₂O₂ (DCF fluorescence)

1. Control neutrophils received neither SPRST nor fMLP treatment.

2. fMLP, samples stimulated with fMLP alone

3. fMLP+SPRST, SPRST (10 μg/ml)-pretreated samples stimulated with fMLP.

FIG. 9 Effects of SPRST, Tet, or Fan on fMLP-induced ROS (O₂^{□-}) production.

Neutrophils (1×10⁶/ml) were incubated at 37° C. for 15 min with hydroethidium (10 μM). After labeling, cells were pretreated with 5-10 μg/ml SPRST or other chemicals for 10 min and stimulated with FMLP (1 μM). Production of O₂^{□-} was then determined 30 min later by flow cytometry.

1. Control neutrophils received neither SPRST nor fMLP treatment.

2. fMLP, samples stimulated with fMLP alone

3. fMLP+SPRST, SPRST (10 μg/ml)-pretreated samples stimulated with fMLP.

FIG. 10 Statistical summaries of fMLP-induced H₂O₂ and O₂^{□-} production in the presence of 5-10 μg/ml of SPRST, Tet, or Fan. The control values are 11.0±0.8 and 10.7±0.4 for DCF (H₂O₂) and EB (O₂^{□-}), respectively. Values are mean±S.E.M.(n=5-8). *P<0.05, as compared to samples receiving fMLP alone for DCF (H₂O₂) or EB (O₂^{□-}), respectively.

1. Control (drug free)

2. fMLP only

3. fMLP and 5 μg/ml SPRST

4. fMLP and 10 μg/ml SPRST

5. fMLP and 5 μg/ml Tet

6. fMLP and 10 μg/ml Tet

7. fMLP and 5 μg/ml Fan

8. fMLP and 10 μg/ml Fan

FIG. 11 Mean time-response curves for SPRST, Tet, or Fan in the inhibition of fMLP-induced intracellular alkalization (pHᵢ). Neutrophils (1×10⁶/ml) were loaded with BCECF-AM (2 μg/ml) for 30 min at 37° C. and washed twice. BCECF-loaded neutrophils were pretreated with 10 μg/ml of SPRST, Tet, or Fan as well as 10 μM of verapamil (Verap) for 10 min at 37° C. After stimulating with fMLP (1 μM), pHᵢ was measured by flow cytometry as described in Materials and Methods at the time as indicated in the figure. Values are mean±S.E.M. (n=5).

1. fMLP only

2. fMLP and verapamil

3. fMLP and Fan

4. fMLP and Tet

5. FMLP and SPRST

FIG. 12 Effects of SPRST, Tet, or Fan on fML-induced changes in intracellular calcium concentration ([Ca²⁺]ᵢ). Neutrophils (2×10⁶/ml) were preloaded with fura 2-AM (5 μM) at 37° C. for 45 min and washed twice with HBSS (calcium free). After drug treatments with 5-10 μg/ml of SPRST, Tet, or Fan as well as 10 μM of verapamil (Verapa) for 10 min, 1 ml cell suspension from each treatment was mixed with equal volume of HBSS (with 2 mM Ca²⁺) and transferred into individual cuvettes. For G-protein study, sample was pretreated with 500 ng/ml of pertussis toxin (PTX) at 37° C. for 2 hrs. Samples were gentle mixed with a micromagnetic stirrer at 37° C. for 5 min before addition of 1 μM of fMLP. [Ca²⁺]ᵢ was measured on a spectrofluorometer as described in Materials and Methods. Net increase in [Ca²⁺]ᵢ was calculated by subtracting control values from respective experimental values (control [Ca²⁺]ᵢ in resting cell was 108±16 nM). Values are mean±S.E.M. (n=4-8). *P<0.05, as compared to samples receiving fMLP alone, respectively.

1. fMLP

2. fMLP and PTX

3. fMLP and verapamil

4. fMLP and SPRST (5 μg/ml)

5. fMLP and SPRST (10 μg/ml)

6. fMLP and Tet (5 μg/ml)

7. fMLP and Tet (10 μg/ml)

8. fMLP and Fan (5 μg/ml)

9. fMLP and Fan (10 μg/ml)

FIG. 13 Effects of SPRST, Tet, or Fan on LTB₄-induced changes in intracellular calcium concentration ([Ca²⁺]ᵢ). Neutrophils (2×10⁶/ml) were preloaded with fura 2-AM (5 μM) at 37° C. for 45 min and washed twice with HBSS (calcium free). After drug treatments with 5-10 μg/ml of SPRST, Tet, or Fan as well as 10 μM of verapamil (Verapa) for 10 min, 1 ml cell suspension from each treatment was mixed with equal volume of HBSS (with 2 mM Ca²⁺) and transferred into individual cuvettes. For G-protein study, sample was pretreated with 500 ng/ml of pertussis toxin (PTX) at 37° C. for 2 hrs. Samples were gentle mixed with a micromagnetic stirrer at 37° C. for 5 min before addition of 0.1 μM LTB₄ (lower panel). [Ca²⁺]ᵢ was measured on a spectrofluorometer as described in Materials and Methods. Net increase in [Ca²⁺]ᵢ was calculated by subtracting control values from respective experimental values (control [Ca²⁺]ᵢ in resting cell was 108±16 nM). Values are mean±S.E.M. (n=4-8). *P<0.05, as compared to samples receiving LTB₄ alone, respectively.

1. LTB₄

2. LTB₄ and PTX

3. LTB₄ and SPRST (5 μg/ml)

4. LTB₄ and SPRST (10 μg/ml)

5. LTB₄ and Tet (5 μg/ml)

6. LTB₄ and Tet (10 μg/ml)

7. LTB₄ and Fan (5 μg/ml)

8. LTB₄ and Fan (10 μg/ml)

FIG. 14. Effects of SPRST, Tet, or Fan on AlF₄⁻-induced changes in intracellular calcium concentration ([Ca²⁺]ᵢ). Fura 2-AM or BCECF-AM labeled-neutrophils were pretreated with 5-10 μg/ml of SPRST or other chemicals at 37° C. for 10 min. For G-protein study, sample was pretreated with 500 ng/ml of pertussis toxin (PTX) at 37° C. for 2 hrs before the addition of AlF₄⁻ (10 mM NaF plus 10 μM AlCl₃), a direct G-protein activator. AlF₄⁻-induced changes in [Ca²⁺]ᵢ were measured as described in Materials and Methods. Untreated neutrophils displayed spontaneous adhesion with a fluorescence intensity of 218±22. Values are mean±S.E.M. (n=5). *P<0.05, as compared to samples receiving AlF₄⁻ alone for [Ca²⁺]ᵢ and neutrophil adhesion, respectively.

1. AlF₄⁻

2. AlF₄⁻ and PTX

3. AlF₄⁻ and SPRST (5 μg/ml)

4. AlF₄⁻ and SPRST (10 μg/ml)

5. AlF₄⁻ and Tet (5 μg/ml)

6. AlF₄⁻ and Tet (10 μg/ml)

7. AlF₄⁻ and Fan (5 μg/ml)

8. AlF₄⁻ and Fan (10 μg/ml)

FIG. 15. Effects of SPRST, Tet, or Fan on AlF₄⁻-induced changes in neutrophil adhesion. Fura 2-AM or BCECF-AM labeled-neutrophils were pretreated with 5-10 μg/ml of SPRST or other chemicals at 37° C. for 10 min. For G-protein study, sample was pretreated with 500 ng/ml of pertussis toxin (PTX) at 37° C. for 2 hrs before the addition of AlF₄⁻ (10 mM NaF plus 10 μM AlCl₃), a direct G-protein activator. AlF₄⁻-induced neutrophil adhesion were measured as described in Materials and Methods. Untreated neutrophils displayed spontaneous adhesion with a fluorescence intensity of 218±22. Values are mean±S.E.M. (n=5). *P<0.05, as compared to samples receiving AlF₄⁻ alone for [Ca²⁺]ᵢ and neutrophil adhesion, respectively.

1. AlF₄⁻

2. AlF₄⁻ and PTX

3. AlF₄⁻ and SPRST (5 μg/ml)

4. AlF₄⁻ and SPRST (10 μg/ml)

5. AlF₄⁻ and Tet (5 μg/ml)

6. AlF₄⁻ and Tet (10 μg/ml)

7. AlF₄⁻ and Fan (5 μg/ml)

8. AlF₄⁻ and Fan (10 μg/ml)

FIG. 16. Effects of various SPRST extracts on superoxide anion (O₂⁻.) production by PMA-stimulated human neutrophils. Samples were pretreated with various SPRST extracts (100 μg/ml) at 37° C. for 10 min. PMA (100 ng/ml)-induced O₂⁻. (EB) production was measured by a flow cytometer (FACSCalibur™) 30 min after addition of PMA (100 ng/ml). *P<0.05 as compared with samples treated with PMA alone. Values are means±S.E.M. from 6 experiments. RST/H₂O, RST extracted by water only; RST/H₂O/EtOH, RST residue extracted by ethanol after water extraction; RST/EtOH, RST extracted by ethanol only; SPRST/EtOH/H₂O, RST residue extracted by water after extraction with ethanol; RST/EtOH/CH₂Cl₂, RST residue extracted by CH₂Cl₂ after extraction with ethanol; RST/CH₂Cl₂, RST extracted by CH₂Cl₂ only; RST/CH₂Cl₂/EtOH, RST residue extracted by ethanol after CH₂Cl₂ extraction; RST/CH₂Cl₂/H₂O, RST residue extracted by water after CH₂Cl₂ extraction.

1. Control (drug free)

2. PMA

3. RST/H₂O

4. RST/H₂O/EtOH

5. RST/EtOH

6. RST/EtOH/H₂O

7. RST/EtOH/CH₂Cl₂

8. RST/ CH₂Cl₂

9. SPRST/CH₂Cl₂/EtOH

10. SPRST/CH₂Cl₂/H₂O

FIG. 17. Effects of Fan, Tet or SPRST on cell viability. Cell viability was measured by a propidium iodide exclusion assay. After incubation of cells (2×10⁶/ml) with SPRST or test drugs for 1 h, cell suspension was further incubated with propidium iodide (10 μg/ml) and fluorescein diacetate (100 ng/ml) at room temperature for 10 min. Cell suspension was analyzed immediately on a flow cytometer (FACSCalibur™; Becton Dickinson) by recording forward and light scatter, red (>630 nm) and green (520 nm) fluorescence. After gating for light scatter to include single cells and to exclude clumps and debris, cell populations (1×10⁴ cells) were displayed as green (viable) versus red (dead) fluorescence. Cell viability (%) was calculated by the CellQuest® software (Becton Dickinson) on a Power Macintosh 7300/200 computer. Values represent the means±S.E.M. of five experiments performed on different days using cells from different rats.

1. Vehicle control (0.5% DMSO)

2. PMA

3. staurosporine

4. Fan 1 μg/ml

5. Fan 5 μg/ml

6. Fan 10 μg/ml

7. Tet 1 μg/ml

8. Tet 5 μg/ml

9. Tet 10 μg/ml

10. SPRST 1 μg/ml

11. SPRST 5 μg/ml

12. SPRST 10 μg/ml

13. Tet (1 μg/ml) +Fan (1 μg/ml)

14. Tet (2.5 μg/ml)+Fan (2.5 μg/ml)

15. Tet (5 μg/ml) +Fan (5 μg/ml)

### DESCRIPTION OF THE PREFERRED EMBODIMENTS

The root of Stephania tetrandra S. Moore was appraised and punchased from Chinese herbal market in Taipei (Taiwan). That is the Chinese traditional medicine Menispermaceae family ‘Fen-Fan-Chi’ plant. The extract of Radix Stephaniae tetrandrae (RST) was extracted with aquar or/and organical mixture solvent. Some extract process using for natural product or Soxhlet extraction, are suitable for our invention. The organical mixture solvent, selected from one or more then one of ethanol (EtOH), dichloromethane (CH₂Cl₂), acetone (acetone). The combined extracted was dried then isolated through column chromatoghapgy. The suitable gel selected from silica gel, Diaion, Sephadex, C-18 for chromatoghapgy. Suitable eluent solution on chromatoghapgy, selected from one or more then one of water and organical mixture solvent, such as methanol (MeOH), ethanol (EtOH), dichloromethane (CH₂Cl₂), acetone (acetone), toluene.

This invention disclosed specially processed extract of S. tetrandrae (SPRST) have pharmacologically with inflammatory responses, inhibition of neutrophil firm adhesion and transmigration, and prevention of cardiovascular disease. Said specially processed extract of S. tetrandrae (SPRST), not only extracted with aquar or/and organical mixture solvent useing for natural product or Soxhlet extraction, but also including various SPRST extracts. Said various RST extracts, such as RST/H₂O/EtOH, RST/EtOH, RST/EtOH/H₂O, RST/EtOH/CH₂Cl₂, RST/CH₂Cl₂, RST/CH₂Cl₂/EtOH, RST/CH₂Cl₂/H₂O. The preparation method of various SPRST extracts are described in detail that RST residue have extracted again by suitable solvent EtOH, H₂O, CH₂Cl₂ and so on. RST/H₂O/EtOH present RST residue extracted by ethanol after water extraction; RST/EtOH, RST extracted by ethanol only. RST/EtOH/H₂O present RST residue extracted by water after extraction with ethanol. RST/EtOH/CH₂Cl₂ present RST residue extracted by CH₂Cl₂ after extraction with ethanol. RST/CH₂Cl₂ present RST extracted by CH₂Cl₂ only. RST/CH₂Cl₂/EtOH present RST residue extracted by ethanol after CH₂Cl₂ extraction. RST/CH₂Cl₂/H₂O present RST residue extracted by water after CH₂Cl₂ extraction.

The main active constituents in Radix Stephaniae tetrandrae are tetrandrine (Tet), fangchinoline (Fan), oblongine (Obl), cyclanoline (Cyc), menisine and menisidine. For ascertained whether aristolochic acid does not contain into SPRST, a HPLC methods was developed.

A variety of solvents were tested for their ability to separate the four alkaloids present in the plant extract. Gradient systems of MeOH—H₂O or MeCN—H₂O in combination with (NH₄)H₂PO₄ buffer on a reverse column (Cosmosil 5C18-AR-II, 4.6×25 mm) did not good in the resolution, with ion-pair reagent (SDS) did not result in the complete separation. Eventually, gradient systems of MeCN—H₂O in combination KH₂PO₄ buffer at pH 2.91-3.00 (this profile described in section 2-4) were used to achieve complete separation on the same reversed-phase column

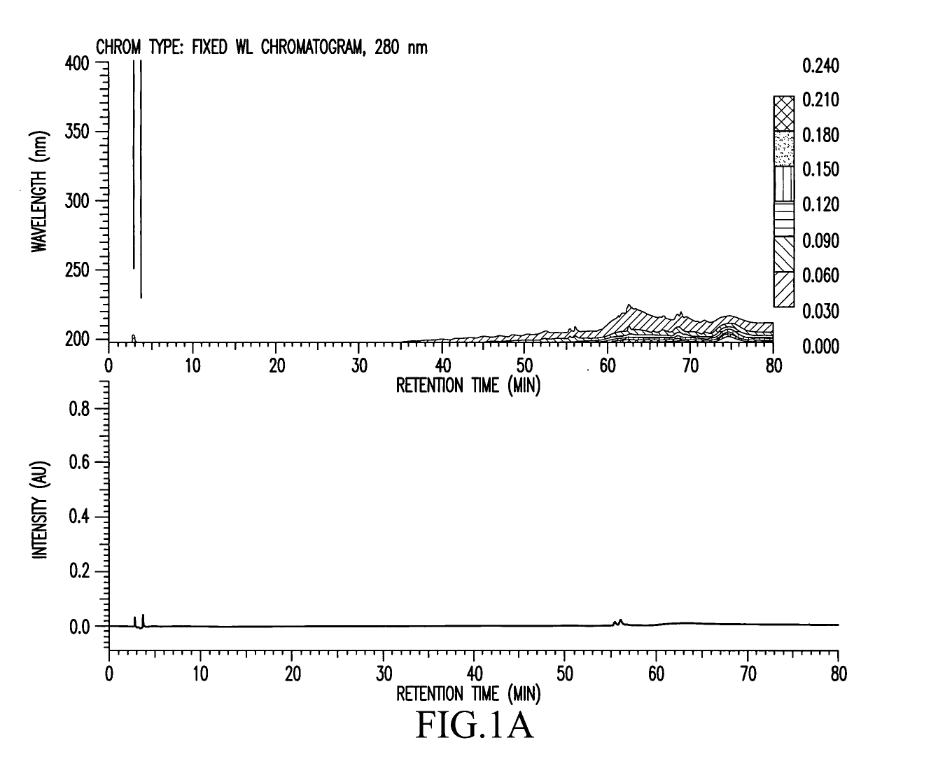

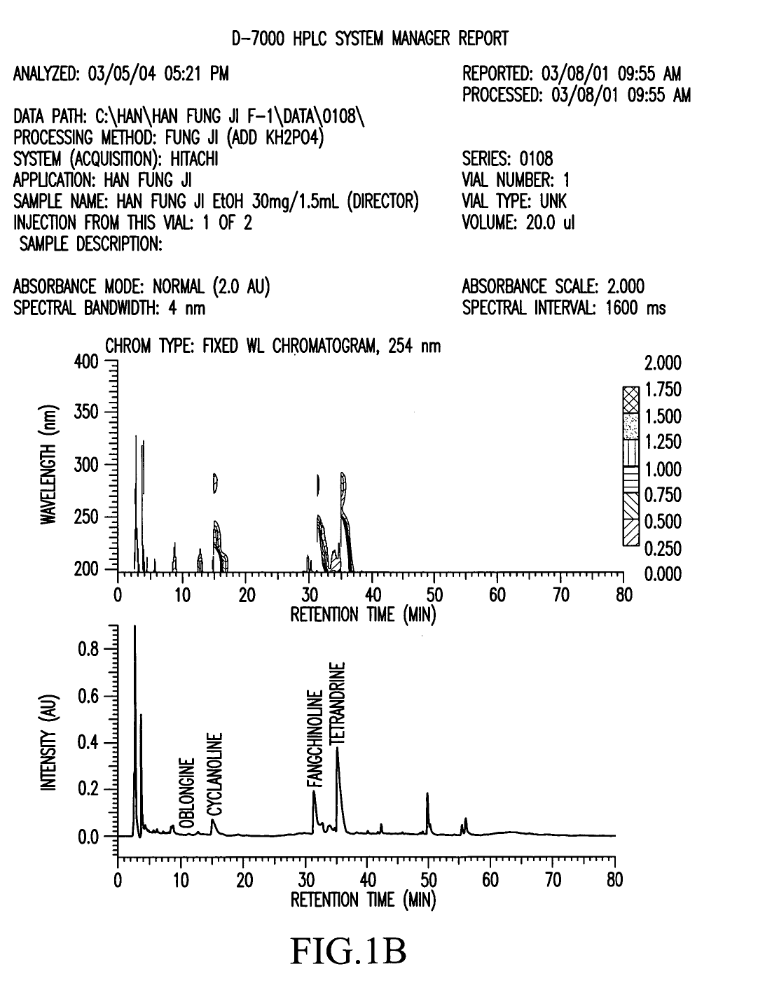

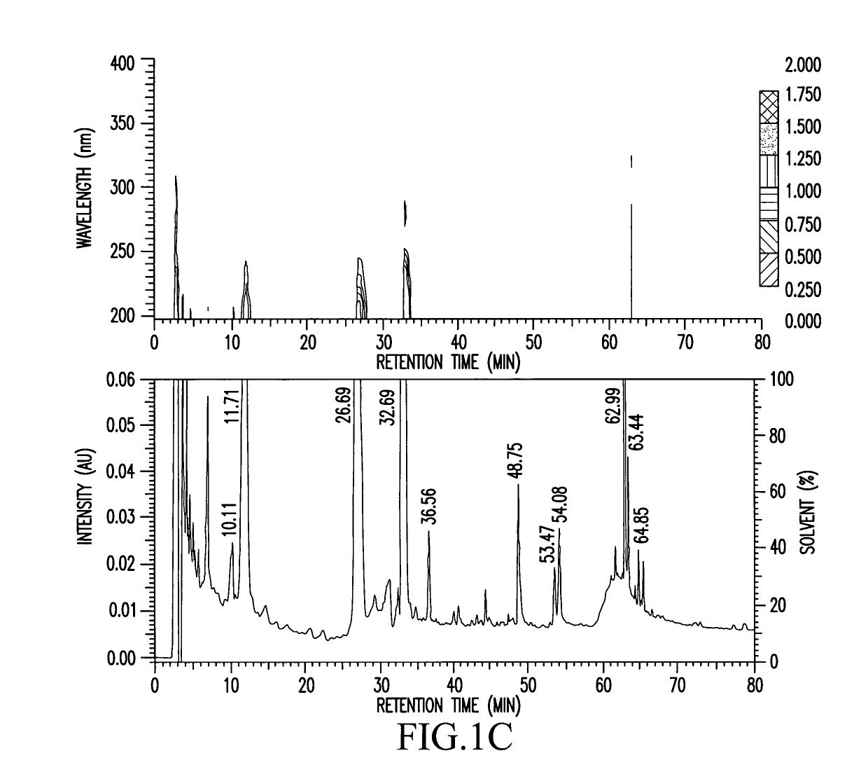

The reversed-phase high-performance liquid chromatographic method to simultaneously measure the four alkaloids in Radix Stephaniae tetrandrae has been successfully developed. The method uses the four compounds as external standards. These compounds were isolated in our laboratory using various chromatographic methods, which are completely separated within 45 min using a reversed-phase column and linear gradient elution with dihydrogenphosphate buffer HPLC-grade acetonitrile mobile phase. The quantitative calibration curves are linear covering a range of 12.5-1637 μg/ml for all four compounds. The detection limits (S/N=3) for tetrandrine, fangchinoline, cyclanoline and oblongine are approximately 0.95, 0.95, 0.95 and 1.69 μg/ml, respectively. FIG. 1B shown HPLC pattern of four alkaloids, such as tetrandrine, fangchinoline, cyclanoline and oblongine in Radix Stephaniae tetrandrae. Converted integration superficial content into content of tetrandrine, fangchinoline, cyclanoline and oblongine, that hold 5˜20% w/w of SPRST full amount.

That also shown the relention time of four alkaloids according to priority, the oblongine (Obl) is 10.11 min., cyclanoline (Cyc) 11.71 min., fangchinoline (Fan) 26.69 min., and tetrandrine (Tet) 32.69 min. The FIG. 1B does not appear pattern of aristolochic acid on SPRST, even till 80 min.

Drawing blank control of solvent on FIG. 1A, there have not any pattern of ingredients during relention time of 5-50 min. After sampled the mixture of aristolochic acid and SPRST, the normal pattern of four alkaloids, and peak of aristolochic acid at relention time of 63.12 min are observed (FIG. 1C).

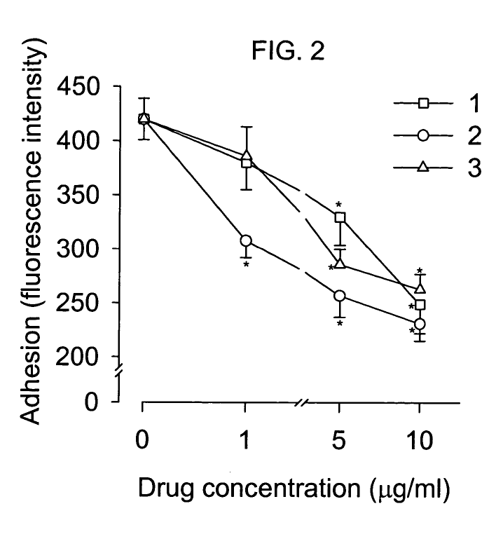

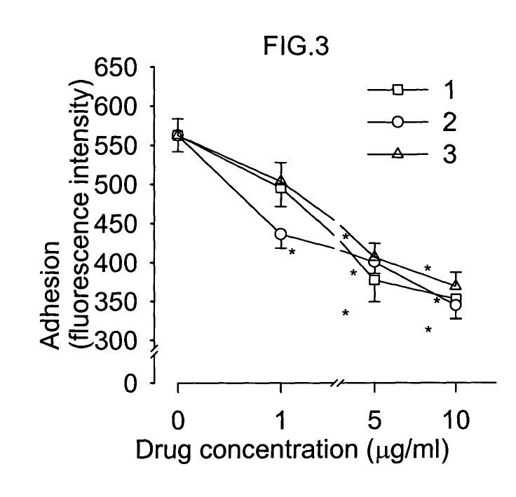

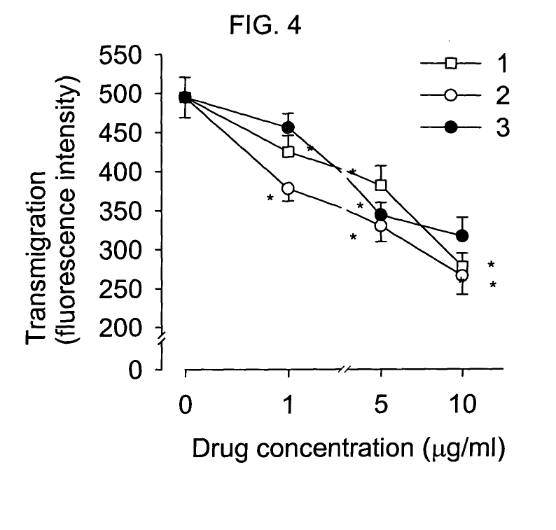

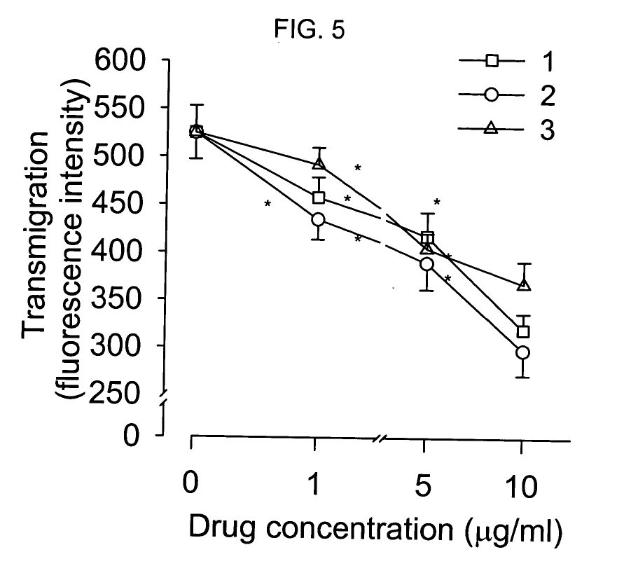

In the invention, neutrophils pretreated with 1-10 μg/ml of SPRST for 10 minutes significantly impaired neutrophil firm adhesion (FIG. 2 & FIG. 3) and transmigration (FIG. 4 & FIG. 5). Tet or Fan, two active components in SPRST, displayed similar efficacy as SPRST (FIG. 2 & FIG. 3). In neutrophils pretreated with 0.1 μg/ml of Tet or Fan (SPRST containing 1.3% Tet and 0.7% Fan), neither single drug treatment nor combination of Tet and Fan could attenuate neutrophil firm adhesion or transmigration (data not shown), indicating that other elements, in addition to Tet and Fan, might be involved in mediating the effect of SPRST. The anti-adhesive and transmigration prevention effects of SPRST were not due to cytotoxicity because under these conditions there was no difference in cell viability between SPRST-treated neutrophils and control cells (viability>95% at the end of the experiments; FIG. 15). To further elucidate the mechanism(s) involved in the anti-inflammatory effects of SPRST, in view of the importance of Mac-1 (CD11b/CD18) in neutrophil adhesion (Albelda et al., 1994) and transmigration (Werr et al., 2000), we further examined the effect of SPRST on cell surface expression levels of Mac-1.

It has been reported that ROS enhanced Mac-1 upregulation and anti-oxidants diminished Mac-1-mediated neutrophil accumulation and adhesion following ischemia and reperfusion; (Serrano et al., 1996; Fraticelli A, Serrano C V J, Bochner B S, Capogrossi M C and Zweier J L, Biochim Biophys Acta 1310:251-259,1996). In this study, ROS (O₂^{□-} and H₂O₂) production induced by fMLP was diminished by SPRST as well as Tet and Fan (FIG. 4). This indicates that SPRST, Tet, and Fan may act as ROS scavengers through which in turn down-regulate Mac-1 expression and then neutrophil firm adhesion/transmigration. Our prior studies confirmed that antioxidants (superoxide dismutase and catalase) significantly down regulated ROS production as well as Mac-1 expression and neutrophil adhesion to fibrinogen (Shen Y C, et al., Eur J Pharmacol 343:79-86.1998). The flow cytometric method used in this study for the measurement of ROS production enabled on-line monitoring of the intracellular accumulation of O₂^{□-} and H₂O₂ in neutrophils. We found accumulation of O₂^{□-} and H₂O₂ began immediately after stimulation (data not shown). Thus, the rapid accumulation of O₂^{□-} and H₂O₂ in response to stimulation and our observation that Mac-1 upregulation could be inhibited by ROS scavengers (Shen et al., 1999) suggests that ROS are early signaling molecules involved in the regulation of neutrophil function. This argument is further intensified by Finkel's observations (Finkel T, Curr Opin Cell Biol 10:248-253, 1998) that ROS can act as second messengers in the activation of ligand-stimulated NF-κB, various protein kinase C (PKC) family members, and mitogen-activated protein kinase (MAPK) as well as tyrosine kinases/phosphatase. Thus, we suggest that ROS could regulate neutrophil functions through second messenger mechanism(s).

ROS production by neutrophil through activation of membrane-bound NADPH oxidase is accompanied by transient cytosolic alkalization to maintain the activity of this enzyme (Henderson L M and Meech R W, J Gen Physiol 114:771-785,1999). In this study, fMLP induced a rapid and intense intracellular alkalization (FIG. 11). (Coakley R J, Taggart C, Canny G, Greally P, O'nell S J and McElvaney N G, Am J Physiol 279:L66-L74,2000) had reported comparable finding. Verapamil as well as SPRST, Tet, and Fan limited the prompt cytosolic alkalization (FIG. 11), indicating a calcium dependent pathway mediated fMLP-induced alkalization that could regulate ROS production. This is further illustrated by the observation that ROS production induced by fMLP is related to calcium-dependent priming of neutrophil which if blocked interferes with ROS production (Lew et al., 1984). We found fMLP and LTB₄ triμger prompt and prominent [Ca²⁺]ᵢ increment, and both could be diminished by SPRST, Tet, and Fan (FIG. 12). Thus, modulation of calcium mobilization could be the possible target by these drugs. To elucidate the possible target by SPRST, AlF₄⁻, a direct G protein activator, induced calcium influx was introduced to contrast the receptor (fMLP or LTB₄)-mediated calcium mobilization. SPRST, Tet, and Fan concentration-dependently impaired AlF₄⁻-induced calcium influx as well as neutrophil adhesion (FIG. 13). Therefore, G protein could be modulated by SPRST. Because SPRST, containing 1.3% Tet and 0.7% Fan, was as potent as Tet and Fan in the inhibition of AlF₄⁻-induced calcium influx and neutrophil adhesion (FIG. 13, ANOVA, P>0.05) indicated component(s) in addition to Tet and Fan mediated the inhibitory effect of SPRST.

In addition to inhibition of ROS production and Ca²⁺ mobilization, SPRST may also inhibit other biochemical pathways that could regulate Mac-1 expression. For example, Mac-1 expression may be regulated by phospholipase A₂, which catalyzes the synthesis of arachidonate, because phospholipase A₂ inhibitors can inhibit the surface expression of Mac-1 (Jacobson P B and Schrier D J, J Immunol 151:5639-5652,1993). Tet has been shown to decrease the production of prostaglandin E₂ and leukotriene C₄/D₄/E₄, downstream metabolites of arachidonate (Teh et al., 1990). It is likely that this biochemical pathway may be targeted by SPRST and in turn Mac-1 expression could be regulated. Furthermore, it is noted that MAPK pathways play central role in regulating a wide range of inflammatory responses including activation of NADPH oxidase (Yamamori et al., 2000), migration of neutrophils (Atta U R, Harvey K and Siddiqui R A, Curr Pharmaceutical Design 5: 241-253.1999) as well as beta₂ integrin expression (Tandon et al., 2000). Whether these biochemical pathways are targets of SPRST in the regulation of Mac-1 dependent neutrophil adhesion and transmigration awaits further research and is currently under investigation in our laboratory.

In conclusion, we have demonstrated that inhibition of neutrophil adhesion and transmigration through suppression of Mac-1 upregulation could account for the cardioprotective effect of SPRST. The inhibitory effect of SPRST on Mac-1 expression could be mediated by down regulation of ROS production and intracellular Ca²⁺ mobilization through, at least in part, G protein modulation. The effect of SPRST per se can attribute to component(s) in addition to Tet and Fan because no significant effect was observed by combination low dose (0.1 μg/ml) of Tet and Fan.

In the invention, as effective anti-inflammatory anti-adhesive and transmigration preventing drugs at pharmacological concentrations (1-10 μg/ml), SPRST, along with its active components Tet and Fan, may be clinically beneficial for the amelioration of ischeaemic reperftision injury by limiting the early phases of neutrophil activation.

SPRST, Tet, and Fan significantly prevented fMLP or LTB₄-induced Mac-1 upregulation revealing that the anti-adhesive and transmigration prevention effects and does-dependently inhibited of these drugs were, at least in part, mediated by inhibition of the Mac-1 upregulation on neutrophil membrane.

SPRST (1-10 μg/ml) concentration-dependently prevented N-formyl-methionyl-leucyl-phenylalanine (fMLP)- or leukotriene B₄ (LTB₄)-induced neutrophil firm adhesion and transmigration. Comparable results were also observed in neutrophils pretreated with fangchinoline (Fan) or tetrandrine (Tet), two active components in SPRST. It has been reported that neutrophil firm adhesion/ transmigration is mainly Mac-1 (CD11b/CD18)-dependent and could be modulated by reactive oxygen species (ROS) production. SPRST, Tet, and Fan diminished FMLP- or LTB4-induced Mac-1 up-regulation and ROS production. That SPRST, Fan, and Tet as well as verapamil impaired fMLP-induced rapid intracellular alkalization, an essential mechanism for neutrophil ROS production, and [Ca²⁺]ᵢ increment suggested that a calcium dependent pathway might be involved. Direct G protein activation by AlF₄⁻ also triggered [Ca²⁺]ᵢ increment and firm adhesion which could be abolished by pertussis toxin and were partially reversed by SPRST, Fan, and Tet. These results reveal that inhibition of neutrophil adhesion and transmigration may account for SPRST's myocardial protective effect. This effect of SPRST may be mediated by component(s) in addition to Tet and Fan because combination of 0.1 μg/ml of Tet and Fan did not mimic the effect of SPRST. We conclude that SPRST exerts anti-inflammatory effects by interfering with ROS production and Ca²⁺ influx through G protein modulation to prevent Mac-1 up-regulation in neutrophil activation.

Cytotocixity assay of SPRST and its active principles Tet and Fan. The concentrations (1-10 μg/ml) of these drugs (SPRST, Fan, Tet) used in this study displayed no significant cytotoxicity to neutrophils (viability after drugs treatment more than 98% by propidium iodide exclusion assay). As shown in FIG. 15, the cytotoxic effect of SPRST, Tet or Fan alone was 0.64±0.14%, 1.24±0.17% or 0.84±0.11% at 10 μg/ml, respectively. The cytotoxic effect induced by SPRST was relative minor than its active principles. Combination of Tet and Fan induced more cytotoxic effect (1.5±0.23%) than that of Tet or Fan alone. In drug free condition, dead cell was around 0.65±0.28%. These results indicate that SPRS was less cytotoxic than Tet or Fan at high concentration.

Relationship between Ca²⁺ influx and SPRST-inhibited neutrophil adhesion. In addition to modulating ROS production, cytosolic calcium fluctuation could also regulate neutrophil migration (Lawson and Maxfield, 1995), and we have previously reported that impediment to calcium influx diminished Mac-1 dependent neutrophil adhesion (Shen et al., 1999); therefore, effects of SPRST, Tet, and Fan in Ca²⁺ mobilization were determined. Calcium influx could be triμgered by receptor-coupled activation or by direct G protein activation. To elucidate the possible targets of these drugs, fMLP/LTB₄ (receptor-mediated) or AlF₄⁻ (direct G protein-mediated) induced calcium mobilization was performed. FMLP or LTB₄ triggered rapid increase in [Ca²⁺]ᵢ which was abolished by pertussis toxin (PTX) pretreatment, and were concentration-dependently inhibited by SPRST, Tet, or Fan (FIG. 12, P<0.05, n=4-8). AlF₄⁻-induced [Ca²⁺]ᵢ increment and neutrophil firm adhesion were also significantly inhibited by PTX, and concentration-dependently decreased by SPRST, Tet, or Fan (FIG. 13, P<0.05, n=5). SPRST was as potent as Tet or Fan in antagonizing Ca²⁺ mobilization or adhesion induced by AlF₄⁻ (FIG. 13, ANOVA, P>0.05).

SPRST and Its Active Components Tet and Fan Inhibit Neutrophil Adhesion and Transmigration. To examine whether SPRST and/or its active components Tet and Fan could inhibit neutrophil infiltration, we established an in vitro assay system in which fMLP (1 μM) or LTB₄ (0.1 μM) was used to induce neutrophil firm adhesion and transmigration, functions underlying neutrophil infiltration. In the adhesion assay, whereas untreated neutrophils displayed spontaneous adhesion with a fluorescence intensity of 206±18, fMLP or LTB₄ caused up to 200% enhancement in neutrophil firm adhesion relative to background levels (FIG. 2 & FIG. 3). Pretreatment of neutrophils with SPRST, Tet, or Fan does-dependently inhibited fMLP- or LTB₄-induced neutrophil firm adhesion (FIG. 2 & FIG. 3). Combination of 1 or 10, but not 0.1 μg/ml Tet and Fan further attenuated neutrophil adhesion (data not shown). Similar results were also observed in the transmigration study (FIG. 4 & FIG. 5). Untreated neutrophils displayed spontaneous transmigration with a fluorescence intensity of 254±14 (FIG. 4). SPRST, Tet, or Fan alone did not influence spontaneous neutrophil adhesion or transmigration (ANOVA, P>0.05). The concentrations of these drugs used in this study were not cytotoxic to neutrophils (viability after drugs treatment >95% by propidium iodide exclusion assay).

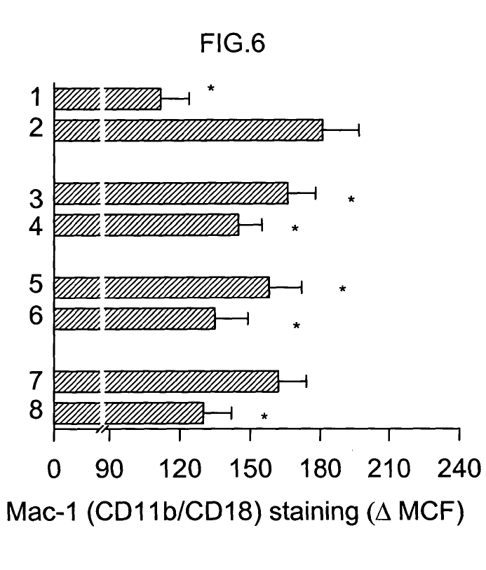

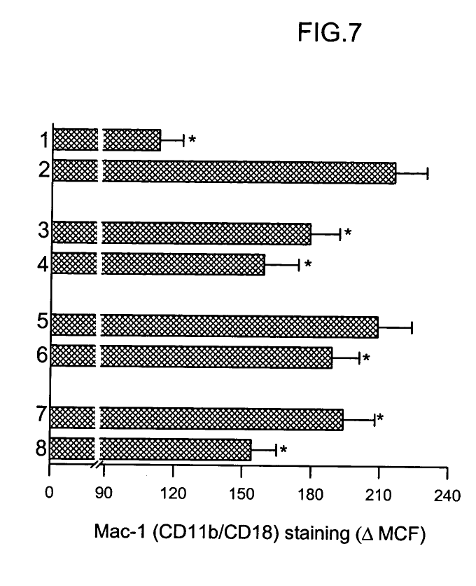

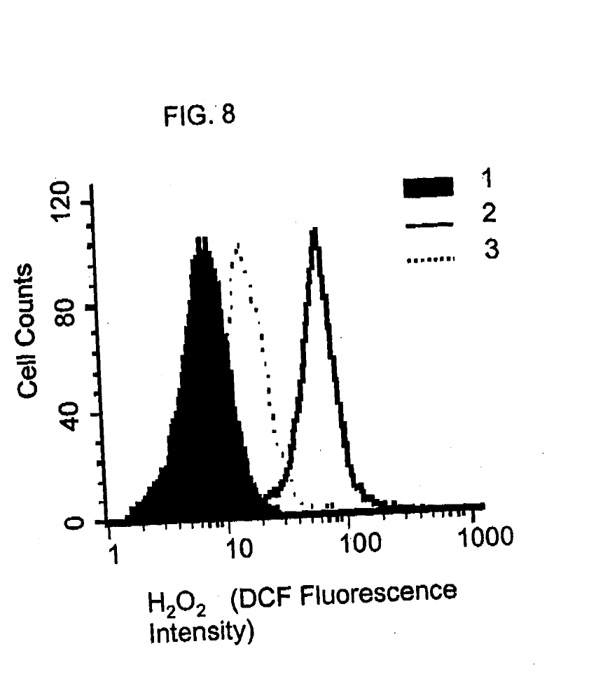

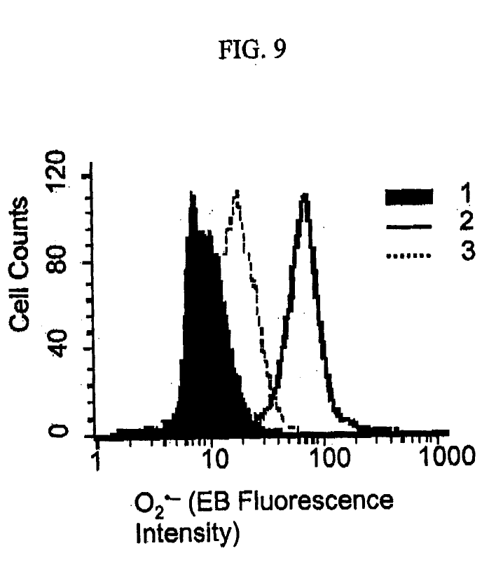

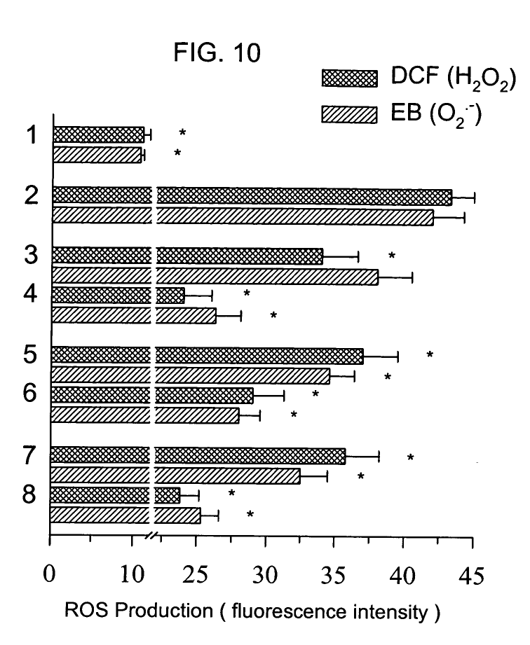

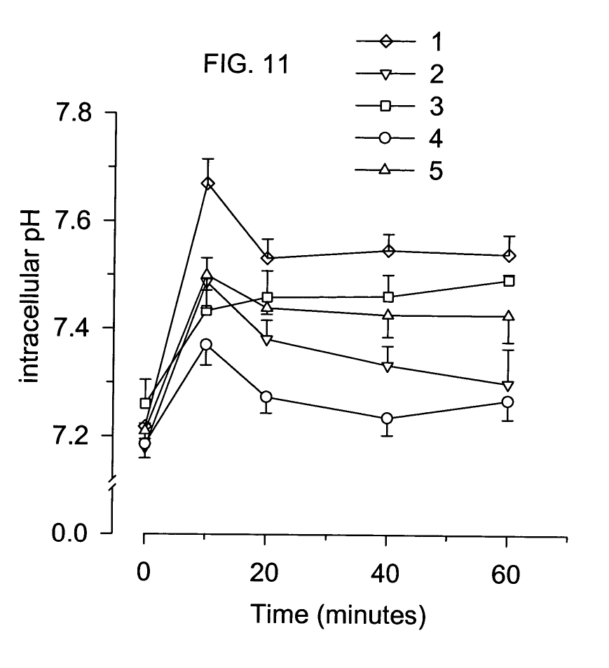

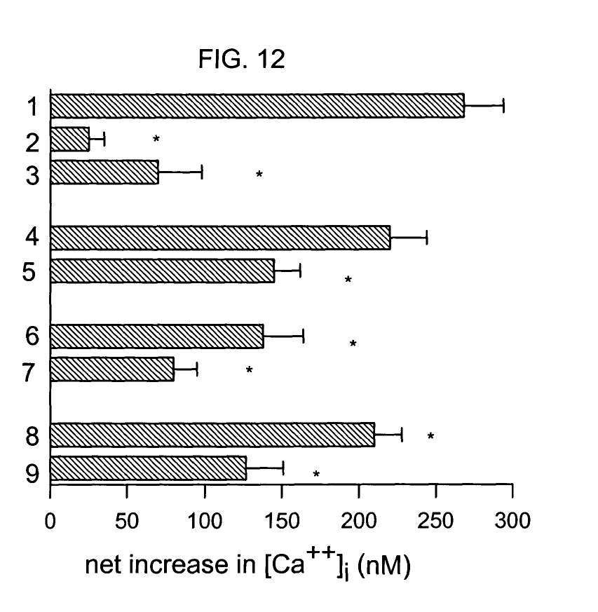

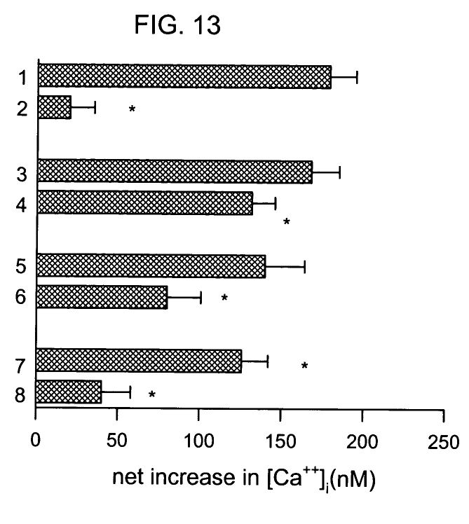

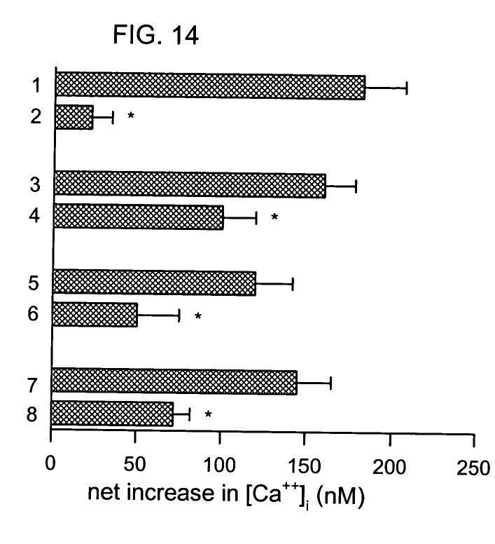

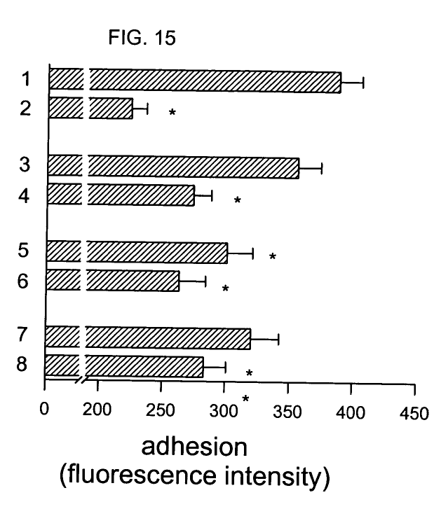

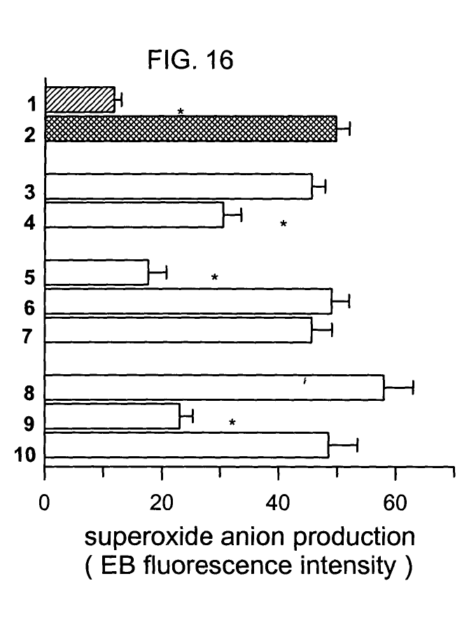

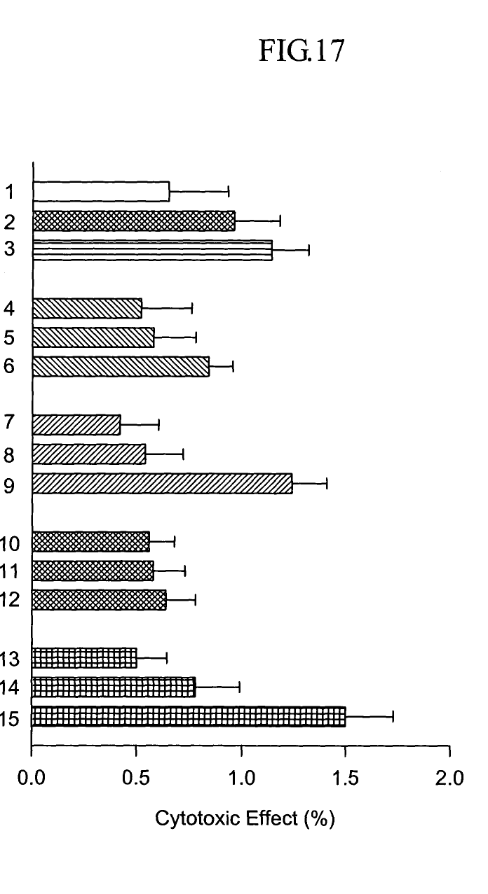

SPRST, Tet, and Fan inhibit Mac-1 (CD11b/CD18) upregulation. Neutrophil adhesion to the extracellular matrix has been shown to mainly depend on upregulation of Mac-1 (CD11b/CD18) (Everitt E A, Malik A B and Hendey B, J Leukoc Biol 60:199-206,1996), and β₂ integrins may serve to regulate neutrophil extravasation (Werr et al., 2000). Therefore, we examined whether SPRST, Tet, or Fan could inhibit neutrophil adhesion and/or transmigration by virtue of down regulation of Mac-1. To assess the effect of these drugs on Mac-1 expression, we measured surface levels of Mac-1 on fMLP- or LTB₄-stimulated neutrophils with or without drug(s) pretreatment by flow cytometric analysis. FMLP or LTB₄ caused a marked increase in Mac-1 fluorescence while an apparent shifting-to-the-left of Mac-1 fluorescence was observed in samples pretreated with SPRST (10 μg/ml) (FIG. 6). A statistical summary revealing Tet and Fan, as well as SPRST significantly inhibited fMLP- or LTB₄-induced Mac-1 upregulation was illustrated in FIG. 7 (P<0.05, n=3-5).

SPRST, Tet, and Fan inhibited intracellular ROS (O₂^{□-} and H₂O₂) production. It has been shown that ROS (e.g., O₂^{□-} and H₂O₂) upregulates Mac-1 expression and enhances neutrophil adhesion that could be abolished by antioxidants (Serrano et al., 1996; Fraticelli et al., 1996). Therefore, we hypothesized the de novo production of ROS by neutrophils may participate in Mac-1 upregulation that could be diminished by SPRST. We used a flow cytometric method to measure intracellular ROS production in fMLP-stimulated neutrophils in the presence or absence of SPRST. A representative experiment by fMLP-stimulated accumulation of intracellular H₂O₂ (measured as DCF fluorescence) and O₂^{□-} (measured as EB fluorescence), respectively, were illustrated in FIG. 8 & FIG. 9 while the results of five experiments are summarized in FIG.10). SPRST, Tet, and Fan concentration-dependently decreased the fluorescence intensity of EB and DCF induced by fMLP (FIG. 10, P<0.05, n=5-8).

SPRST, Tet, and Fan limited fMLP-induced intracellular pH (pHᵢ) alkalization. ROS production induced by fMLP is a calcium sensitive event (Lew et al., 1984) and accompanied by transient cytosolic alkalization to maintain the activity of NADPH oxidase (Henderson and Meech, 1999). In this study, we observed that fMLP induced a rapid and profound alkalization of pHᵢ over 60 min (FIG. 11). Pretreatment with 10 μg/ml of SPRST, Tet, or Fan as well as verapamil (10 μM) significantly limited the cytosolic alkalization induced by fMLP (ANOVA, P<0.05, n=5) indicating that these drugs may modulate a calcium-dependent pathway.

In summary, this invention is an original idea that provides a perspective and novel thinking process for the following considerations: (1) extraction of SPRST is more simple and lower-costed with high-yielding rate than that of extraction of acive components Tet and Fan from plant (2) SPRST is relative lower cytotoxic than its active components Tet or Fan at the same pharmacological applicable concentrations, (3) using HPLC, it is very convenient and fast to identify and quantify the active component Tet or Fan in theSPRST, thus providing a good quality control for chemical fingerprints (4) using flow cytometry to confirm the bio-activities of these active components of SPRST in the anti-inflamamtory effects shows convincing and promising results, and (5) double checking the quality of SPRST by chemical fingerprints and our bio-assay system provides a excellent method for the quality control of our intervention and fits the high standard of drug product manufacturing.

The active components of Tet, Fan and other components in SPRST of this invention will include various excipients; carriers or diluents and pharmaceutically approved pH of processed salts in accordance to necessity to form composition with therapeutic efficacy. Such pharmaceutical preparation could be in solid form for oral and rectum administration; liquid form or non-intestinal injection form; or ointment form for direct application on affected part. Such solid forms are manufactured according to common pharmaceutical preparation methods, which will include disintegrant like starch; sodium carboxymethyl cellulose, adhesive like ethanol; glycerine, or magnesium stearic acid; lactose to make into pharmaceutical preparation like tablets or filled into capsules or suppository. Solution or saline that include this invention compound as ingredient could use buffers of phosphoric nature to adjust the pH to suitable level, before adding adjutant; emulsifier to produce injection dose or other liquid preparation. This invention compound or pharmaceutical manufacturing could mix synthetic acid salts with various fundamental preparations to form ointments according to known pharmaceutical manufacturing methods. Pharmaceutical compounds manufactured with this invention compound being the major ingredient could be used on mammals to produce the efficacy of this main ingredient. General dosage could be adjusted according to the degree of symptoms, and normally a person will require 50 to 300 mg each time, three times per day.

### EXAMPLE 1

Radix Stephaniae tetrandrae (100 g) was milled and extracted with 95% EtOH three times at 80□ (each 1000 ml, 8 h). The combined extract was concentrated by rotary evaporation in vacuum at 50□ to dryness.

### EXAMPLE 2

Radix Stephaniae tetrandrae (100 g) was milled and extracted with dichloromethane three times at 80□ (each 1,000 ml, 8 h). Then extracted with MeOH at 80□ (1,000 ml, 8 h). The combined extract was concentrated by rotary evaporation in vacuum at 50□ to dryness.

### EXAMPLE 3

Radix Stephaniae tetrandrae (100 g) was milled and extracted with 95% EtOH two times at 80□ (each 1,000 ml, 8 h). Then extracted with MeOH at 80□ (1000 ml, 8 h).The combined extract was concentrated by rotary evaporation in vacuum at 50□ to dryness.

### EXAMPLE 4

Radix Stephaniae tetrandrae (100 g) was milled and extracted with 95% EtOH two times at 80□ (each 1,000 ml, 8 h). Then extracted with dichloromethane at 80□ (1,000 ml, 8 h). The combined extract was concentrated by rotary evaporation in vacuum at 50□ to dryness.

### EXAMPLE 5

Radix Stephaniae tetrandrae (610 g) was milled and extracted with 95% EtOH three times at 80□ (each 1,000 ml, 8 h). The combined extract was concentrated by rotary evaporation in vacuum at 50□ to dryness. 3% HCl (200 ml) solution was added to the residue, then extracted with CHCl₃ (200 ml×3).

RST extracted by water only; RST/H₂O/EtOH, RST residue extracted by ethanol after water extraction; RST/EtOH, RST extracted by ethanol only; SPRST/EtOH/H₂O, RST residue extracted by water after extraction with ethanol; RST/EtOH/CH₂Cl₂, RST residue extracted by CH₂Cl₂ after extraction with ethanol; RST/CH₂Cl₂, RST extracted by CH₂Cl₂ only; RST/CH₂Cl₂/EtOH, RST residue extracted by ethanol after CH₂Cl₂ extraction; RST/CH₂Cl₂/H₂O, RST residue extracted by water after CH₂Cl₂ extraction.

### EXAMPLE 6

Reagents and Materials

HPLC-grade acetonitrile was purchased from Tedia (Ohio, USA), sodium dihydrogenphosphate, phosphoric acid and silica gel from Merck (Germany). Sephadex LH-20 was purchased from Pharmacia Biotech (Uppsala, Sweden). Water was purified by a Milli Q system from Millipore (Milford, Mass., USA). Radix Stephaniae tetrandrae was purchased from the Chinese herbal market in Taipei (Taiwan). The four standard alkaloids, tetrandrine, fangchinoline, cyclanoline and oblongine were isolated from the roots using chromatographic methods described below.

Isolation of Four Standard Alkaloids

Radix Stephaniae tetrandrae (610 g) was milled and extracted with 95% EtOH three times at 80□ (each 1000 ml, 8 h). The combined extract was concentrated by rotary evaporation in vacuum at 50□ to dryness. 3% HCl (200 ml) solution was added to the residue, then extracted with CHCl₃ (200 ml×3). The acid solution was adjusted to pH 9 with 25% NH₄OH and the resultant suspension were extracted with CHCl₃. The CHCl₃ layer was evaporated to give tetrandrine and fangchinoline. The NH₄OH layer was then partitioned with n-BuOH. The n-BuOH layer was concentrated and a residue (5.1 g) was chromatographed on Sephadex LH-20 column with MeOH to give three fractions (I, II, III). Fraction II, which contained mostly cyclanoline, was recrystallized with MeOH to give cyclanoline as gray-white power. The mother liquid of fraction II and fraction III were combined and were subjected to column chromatography over silica gel eluting with CHCl₃—MeOH (9:1) to afford cyclanoline and oblongine. The four alkaloids were identified by comparing the IR, MS, ¹H- and ¹³C-NMR spectral data with the literature data.^{4, 14-15.}

Preparation of Sample Solution

A 100 g pulverized Radix Stephaniae tetrandrae was extracted five times with EtOH (1,500 ml, successively) by reflux at 80□, each 5 h. The extracts were combined and filtered, then evaporated in vacuum at ca. 50□ to give a 10.23 g of residue. A 30 mg of dried extract was dissolved in 1.5 ml of MeOH. The solution was filtered through a 0.45 μm syringe filter (Gelman Sciences, Ann Arbor, Mich., USA) before use.

Apparatus and Conditions

HPLC was performed on a Hitachi Model L-7100 Intelligent pump system equipped with a Hitachi Model L-7000 interface, a Hitachi Model L-7450A photodiode array detector and a Hitachi Model L-7200 auto-sampler. Detector was set at 280 nm. The separations were obtained with a reversed-phase colurn (Cosmosil 5C18-AR-II, 4.6×250 mm, Kyoto, Japan) eluted at a rate of 1 ml/min with a linear solvent gradient of A and B [A=KH₂PO₄-H₂O, 1000 ml: 5 g, pH was adjusted with 8.5% H₃PO₄ to 2.91; B=H₂O —CH₃CN—KH₂SO₄, 400 ml : 600 ml: 5 g, pH was adjusted with 8.5% H₃PO₄ to 3.30] according to the following profile: 0-20 min, 72% A, 28% B; 20-55 min, 72-30% A, 28-70% B; 55-60 min, 30%-0% A, 70%-100% B; 60-80 min, 0% A, 100% B; 80-85 min, 0-72% A, 100-28% B; 85-100 min, 72% A, 28% B. The injected volume was 20 μl of the preparative solution.

Preparation of Standard Solution and Calibration

To prepare a standard solution, an accurately weighed amount of the four standard alkaloids, tetrandrine, fangchinoline, cyclanoline and oblongine were dissolved in MeOH. Calibration curves were established based on five points covering a concentration range of 12.5-250 μg/ml for tetrandrine, 12.5-250 μg/ml for fangchinoline, 163.7-1637.5 μg/ml for cyclanoline, 145-1450 μg/ml for oblongine. The standard solution (20 μl) were used for HPLC injections (n=5). Calibration graphs were plotted subsequent to linear regression analysis of the peak area with concentrations.

Preparation of Recovery Studies

Three different concentrations of standard alkaloids; 708, 683 and 593 μg/ml for tetrandrine, 398, 373 and 360 μg/ml for fangchinoline, 800, 636 and 571 μg/ml for cyclanoline, 307, 168 and 110 μg/ml for oblongine were added to each sample solution, respectively. All samples were filtered through a 0.45 μm syringe filter (Gelman) and injected for HPLC analysis to calculate the concentration of tetrandrine, fangchinoline, cyclanoline and oblongine from their calibration graphs.

### EXAMPLE 7

### Tablet Dosage Form

| | |
|---|---|
| SPRST | 50 mg |
| lactose | 30 mg |
| starch | 4 mg |
| magnesium stearate | 6 mg |
| corn starch | 10 mg |

### Materials and Methods

Human Neutrophils Isolation. Preparation of human neutrophils was obtained by venipuncture from adult healthy volunteers and collected into syringes containing heparin (20 U/ml blood). Neutrophils were isolated by the Ficoll gradient centrifugation method, followed by lysis of contaminating erythrocytes. Briefly, blood samples were mixed with an equal volume of 3% dextran solution in a 50-mi centrifuge tube and incubated in an upright position for 30-40 min at room temperature to allow sedimentation of erythrocytes. The upper, leukocyte-rich layer was then collected and subjected to centrifugation at 250×g for 15 min at 4° C. After centrifugation, the pellet was resuspended immediately in a volume of phosphate-buffered saline (PBS) equal to the starting volume of blood. The cell suspension was then apportioned, 6 ml per tube, into 15-ml centrifuge tubes, followed by laying 8 ml of 1.077 g/ml Ficoll solution (Histopaque 1077; Sigma Chemicals Co., St. Louis, Mo., USA) beneath the cell suspension, using a pipette. After centrifugation at 400×g for 40 min at 20° C. without brake, the upper (PBS) and lower (Ficoll) layers were carefully removed, leaving the granulocyte/erythrocyte pellet. To remove residual erythrocytes, the pellet was resuspended in 10 ml cold lysis buffer (155 mM NH₄Cl, 10 mM KHCO₃, and 0.1 mM ethylenediaminetetraacetate (EDTA), pH 7.4). The remaining neutrophils were then pelleted, washed twice with ice-cold PBS, and resuspended in an adequate volume of ice-cold Hanks' buffered saline solution (HBSS) until further manipulation. The preparation contained more than 95% neutrophils, as estimated by counting 200 cells under a microscope after Giemsa staining (Sigma). In all cases except the indicated where neutrophils were pretreated with SPRST, Tet, or Fan, the cells were mixed with drug(s) at concentrations ranging from 1 to 10 μg/ml in HBSS for 10 min at 37° C.

Human Neutrophils Isolation. Preparation of human neutrophils was obtained by venipuncture from adult healthy volunteers and collected into syringes containing heparin (20 U/ml blood). Neutrophils were isolated by the Ficoll gradient centrifugation method, followed by lysis of contaminating erythrocytes. Briefly, blood samples were mixed with an equal volume of 3% dextran solution in a 50-ml centrifuge tube and incubated in an upright position for 30-40 min at room temperature to allow sedimentation of erythrocytes. The upper, leukocyte-rich layer was then collected and subjected to centrifugation at 250×g for 15 min at 4° C. After centrifugation, the pellet was resuspended immediately in a volume of phosphate-buffered saline (PBS) equal to the starting volume of blood. The cell suspension was then apportioned, 6 ml per tube, into 15-ml centrifuge tubes, followed by laying 8 ml of 1.077 g/ml Ficoll solution (Histopaque 1077; Sigma Chemicals Co., St. Louis, Mo., USA) beneath the cell suspension, using a pipette. After centrifugation at 400×g for 40 min at 20° C. without brake, the upper (PBS) and lower (Ficoll) layers were carefully removed, leaving the granulocyte/erythrocyte pellet. To remove residual erythrocytes, the pellet was resuspended in 10 ml cold lysis buffer (155 mM NH₄Cl, 10 mM KHCO₃, and 0.1 mM ethylenediaminetetraacetate (EDTA), pH 7.4). The remaining neutrophils were then pelleted, washed twice with ice-cold PBS, and resuspended in an adequate volume of ice-cold Hanks' buffered saline solution (HBSS) until further manipulation. The preparation contained more than 95% neutrophils, as estimated by counting 200 cells under a microscope after Giemsa staining (Sigma). In all cases except the indicated where neutrophils were pretreated with SPRST, Tet, or Fan, the cells were mixed with drug(s) at concentrations ranging from 1 to 10 μg/ml in HBSS for 10 min at 37° C.

Measurement of Neutrophil Firm Adhesion. Adhesion of neutrophils to extracellular matrix was determined in 24-well tissue culture plates (FALCON®, NJ, USA) coated with fibrinogen as our previous study (Shen et al., 1999). Prior to the addition of neutrophils, the plates were incubated with 250 μl per well of human fibrinogen (50 pg/ml in PBS; Chemicon International, Inc., CA) for 2 hrs at 37° C. The wells were washed once with HBSS, blocked with 1% BSA (Sigma, USA) in HBSS for 1 hr at 37° C., and washed twice with HBSS containing 0.1% Tween-20 (Sigma, USA) and once with HBSS. Immediately prior to addition to the coated-plate, neutrophils (1×10⁷ cells/ml) were loaded with 1 μM 2′,7′-bis-(2-carboxyethyl)-5(and-6)-carboxyfluorescein, acetoxymethyl ester (BCECF-AM) (Molecular Probe, Inc., Eugene, Oreg.) in HBSS for 20 min at 37° C. and then washed twice with 10 ml HBSS without Mg²⁺ or Ca²⁺. Two hundred microliters per well of drug-pretreated BCECF-AM labeled neutrophils (5×10⁵ cells/ml in HBSS) was then added to individual wells. After stimulation with fMLP (1 μM) or LTB₄ (0.1 μM) for 15 min at 37° C., non-adherent cells were removed by aspiration and the wells were gently washed twice with warm PBS containing 1 mM Ca²⁺. Adherent neutrophils were then determined by measuring the fluorescence with a fluorescent plate reader (Cytofluor 2300, Millipore®) with excitation at 485 nm and emission at 530 nm. Data are expressed as fluorescence intensity.

Measurement of Neutrophil Transmigration. Transmigration of neutrophils was quantified as described previously (Krull M, et al., J Immunol 162, 4834-4841, 1999) with some modification. Briefly, 6.5-mm-diameter Transwell inserts of 5 Jm pore size (Coming Costar, Cambridge, Mass., USA) were pre-coated with human fibrinogen (20 μg/ml, 100 μl). Immediately prior to add to the upper chamber of fibrinogen-coated inserts, one hundred microliters per well of BCECF-labeled neutrophils (5×10⁵ cells /ml in HBSS) were treated with SPRST, Tet, or Fan for 10 min at 37° C. Then, FMLP (1 μM) or LTB4 (0.1 μM) was added to the lower chambers and incubated with cells in the upper inserts for 60 min at 37° C. Fluorescence intensity in the lower chambers (represent migrated neutrophils) was quantitated with a fluorescent plate reader (Cytofluor 2300, Millipore®) with excitation at 485 nm and emission at 530 nm. Data are expressed as fluorescence intensity.

Measurement of Mac-1 Upregulation by Flow Cytometry. Expression of Mac-1 (CD11b/CD18) was analyzed as our previous study (Shen et al., 1999). Briefly, SPRST-pretreated neutrophils were stimulated with FMLP (1 μM) or LTB₄ (0.1 μM) for 15 min. The cells were then pelleted and resuspended in 1 ml ice-cold PBS containing 10% heat-inactivated fetal bovine serum (FBS) and 10 mM sodium azide. For staining of Mac-1, all subsequent steps were carried out in an ice bath. Cells were incubated in the dark for 60 min with a proper aliquot of fluorescein isothiocyanate (FITC)-conjugated anti-Mac-1 antibody (mouse anti-human CD11b, class IgG₁; Pharmingen, San Diego, Calif.) or a non-specific mouse antibody (class IgG₁, Sigma) as a negative control. After two washes with PBS containing 5% FBS, stained cells were resuspended in flow cytometer sheath fluid (Becton Dickinson) containing 1% of paraformaldehyde and analyzed on a flow cytometer (FACSort; Becton Dickinson) for Mac-1 expression. Data are expressed as mean channel fluorescence for each sample as calculated by the CellQuest® software (Becton Dickinson) on a Power Macintosh 6100/66 computer.

Flow cytometric Analysis of Intracellular ROS Production. Intracellular production of O₂^{□-} and H₂O₂ were measured as ROS production in this study and analyzed on a flow cytometer (FACSort; Becton Dickinson) according to our previous work (Shen et al. 1998). Briefly, neutrophils (1×10⁶ cells/ml) were incubated at 37° C. for 5 min with 20 μM 2′,7′-dichlorofluorescin diacetate (DCFH-DA; Molecular Probes, Inc., Eugene, Oreg.) and for an additional 15 min with 10 μM of hydroethidine (Molecular Probes). The acetate moieties of DCFH-DA are cleaved off intracellularly by esterases, liberating the membrane impermeable 2′,7′-dichlorofluorescin, which fluoresces when oxidized to 2′,7′-dichlorofluorescein (DCF) by H₂O₂; hydroethidium, on the contrary, can be directly oxidized by O₂^{□-} to ethidium bromide (EB), which fluoresces after intercalating with nucleic acids. After labeling, cells were pretreated with SPRST or other chemicals for 10 min and stimulated with fMLP (1 μM). Production of O₂^{□-} and H₂O₂ was then determined 30 min after on a flow cytometer (FACSort; Becton Dickinson) by measuring emission at 525 nm (FL1) for DCF and 590 nm (FL2) for EB. Data are expressed as mean channel fluorescence.

Determination of Intracellular Calcium Concentration ([Ca²⁺]ᵢ).

Prior to drug treatment, neutrophils were preloaded with 5 μM of 1-[2-(5-carboxyoxal-2-yl)-6-amino-benzofuran-5-oxyl]-2-(2′-amino-5′-m ethylphenoxy-ethane-N,N,N′,N′-tetraacetic acid acetoxymethyl ester (fura 2-AM, Molecular Probes, Eugene, Oreg.) at 37° C. for 45 min, washed twice and resuspended at 2×10⁶ cells/ml in calcium free HBSS containing SPRST, Tet, Fan, or control vehicle. After drug treatment for 10 min, 1 ml cell suspension from each sample and 1 ml HBSS containing 2 mM Ca²⁺ were transferred to individual cuvettes and gently mixed with a micromagnetic stirrer at 37° C. for 5 min before addition of fMLP (1 μM), LTB₄ (0.1 μM), or AlF₄⁻ (10 mM NaF plus 10 μM AlCl₃). The fluorescence of fura-2-loaded cells was measured on a spectrofluorometer (Hitachi F-4500) with excitation at 340 and 380 nm and emission at 510 nm. Intracellular calcium concentration for each sample was calculated from the ratio of emission versus excitation as previously described (Shen et al., 1999):

[Ca²⁺]ᵢ=K.(R−Rₘᵢₙ)(S_{f380})/(Rₘₐₓ−R)(S_{b380})

Where: K=224 nM (Fura-2 at 37° C.), Rₘᵢₙ=ratio value in minimal Ca²⁺ conditions, Rₘₐₓ=ratio value at a maximal Ca²⁺ concentration, S_{f380}=380 nm reading in minimal Ca²⁺ conditions (corrected for background), S_{b380} =380 nm reading in maximal Ca²⁺ conditions (corrected for background). Rₘₐₓ and S_{b380} were obtained at the end of a measurement by permeabilizing the cells with 0.2% digitonin, where Rₘᵢₙ and S_{f380} were determined by adding 20 mM EGTA after digitonin lysis. All measurements were performed in Ca²⁺-containing medium, because no significant changes in [Ca²⁺]ᵢ could be detected under Ca²⁺-free conditions.

Determination of Intracellular pH (pHᵢ). The method described by Boyer & Hedley (1994) was followed. Briefly, cells were loaded with BCECF-AM (2 μg/ml) at 37° C. for 30 min, washed twice and resuspended at 1×10⁶ cells/ml in HBSS. After pretreatment with drug(s) for 10 min, fMLP (1 μM) was added to cells suspension and incubated at 37° C. in 5% CO₂ incubator. Samples were measured by flow cytometry (FACSort, Becton Dickinson) at the time as indicated in the figure. Fluorescence intensity of BCECF at 525-535 nm is pH dependent with greater intensity at higher pH. In order to make measurements of pHᵢ, a ratio was taken between a pH-dependent fluorescence intensity at 525 nm (FL1) and a pH-independent fluorescence intensity at 640 nm (FL3). The value obtained is therefor independent of the factors as photobleaching, cell thickness, and instrument stability as well as nonuniform loading or leakage of the dye. For calibration samples, the pellet was resuspended in high [K⁺] buffers as made by mixing appropriate vulumn of solution 1 (130 mM KH₂PO₄, 20 mM NaCl) and solution 2 (110 mM K₂HPO₄, 20 mM NaCl) to give buffers with a range of known pH between 6.5 and 7.8. Two to three min prior to measurement of pHi of calibration samples, 1 μg/ml nigericin (Sigma), a H⁺/K⁺ ionophore, was added to allow the ratios of intracellular to extracellular potassium ion concentration ([K⁺]ᵢ and [K⁺]ₑ) and that of intracellular to extracellular hydrogen ion concentration ([H⁺]ᵢ and [H⁺]ₑ) to become equal; i.e.:

[K⁺]ᵢ/[K⁺]ₑ=[H⁺]ᵢ/[H⁺]ₑ

For if [K⁺]ᵢ and [K⁺]ₑ are equal, then [H⁺]ᵢ will be equal to [H⁺]ₑ, and hence pHᵢ can be estimated simply by measuring pHₑ. A calibration curve of fluorescence ratio to pH was performed for each experiment individually over a pH range of 6.5-7.8. Data are expressed as pH value of individual samples.

Estimation of cell viability. Cell viability was determined according to Ormerod's method (Ormerod, M. G., 2000. Further applications to cell biology. In: Ormerod, M. G (Ed.), Flow cytometry, Third edition. Oxford University Press, UK, pp. 249-250) after incubation of, cells (2×10⁶/ml) with test drugs for 1 h in 5-ml polystyrene round-bottomed tube (FALCON, Becton Dicson). This method can be adapted for a flow cytometer by adding propidium iodide (10 μg/ml), which is excluded by viable cells but which, when taken up by dead or dying cells, binds to nucleic acids and fluoresces red. The viable cells can be further identified by the addition of fluorescein diacetate (100 ng/ml), which is not fluorescent and which is taken up by cells and is converted to fluorescein by an intracellular esterase. Fluorescein is retained by the cell if the plasma membrane is intact. After incubation with test drugs, cells suspension were further incubated with propidium iodide and fluorescein diacetate at room temperature for 10 min and analyzed immediately on a flow cytometer (FACSCalibur™; Becton Dickinson) by recording forward and light scatter, red (>630 nm) and green (520 nm) fluorescence. After gating for light scatter to include single cells and to exclude clumps and debris, cell populations were displayed by green (viable) versus red (dead) fluorescence. Cell viability (%) was calculated by CellQuest® software (Becton Dickinson) on a Power Macintosh 7300/200 computer. Alternatively, cell viability was further compared by using a cytotoxicity detection kit (Roche®, Germany). This kit measures cytotoxicity and cell lysis by detecting lactate dehydrogenase (LDH) activity released from damaged cells.

SPRST and Other Chemicals

SPRST was prepared as described in previous report (Chou et al., 2002). It was first dissolved in DMSO as a stock solution (20 mM) and then serially diluted in PBS immediately prior to experiments. Stock solution was used within 1 week after preparation. For examination of the effect of these drugs, 10 μl of drug solution was added to 1.0 ml of neutrophil suspension and incubated at 37□ for 10 min prior to the addition of 100 ng/ml PMA (Sigma, USA) or 1 μM fMLP (Sigma, USA). Other chemicals, except where indicated, were purchased from Sigma (USA).

RST/H₂O, RST extracted by water only; RST/H₂O/EtOH, RST residue extracted by ethanol after water extraction; RST/EtOH, RST extracted by ethanol only; RST/EtOH/H₂O, RST residue extracted by water after extraction with ethanol; RST/EtOH/CH₂Cl₂, RST residue extracted by CH₂Cl₂ after extraction with ethanol; RST/CH₂Cl₂, SPRST extracted by CH₂Cl₂ only; RST/CH₂Cl₂/EtOH, RST residue extracted by ethanol after CH₂Cl₂ extraction; RST/CH₂Cl₂/H₂O, RST residue extracted by water after CH₂Cl₂ extraction.

Statistical analysis. All values in the text and figures are given as mean±S.E.M. Parametric data were analyzed by analysis of variance (ANOVA) followed by post-hoc Dunnett's t-test for multiple comparisons. Values of P<0.05 were considered significant.

I claimes

1. The specially processed extract of Radix Stephaniae tetrandrae (SPRST) contains tetrandrine (Tet), fangchinoline (Fan), cyclanoline (Cyc) oblongine (Obl) alkaloids and other compounds with biological activity.

2. A assay specially processed extract of Radix Stephaniae tetrandrae (SPRST) methods,
Standard solution, an accurately weighed amount of the four standard alkaloids, tetrandrine, fangchinoline, cyclanoline and oblongine were dissolved in MeOH;
Calibration curves were established based on five points covering a concentration range of 12.5-250 μg/ml for tetrandrine, 12.5-250 μg/ml for fangchinoline, 163.7-1637.5 μg/ml for cyclanoline, 145-1450 μg/ml for oblongine;
Standard solution (20 μl) were used for HPLC injections (n=5). Calibration graphs were plotted subsequent to linear regression analysis of the peak area with concentrations.

3. The specially processed extract of Radix Stephaniae tetrandrae (SPRST) is a pharmaceutical compound-mixture with anti-inflammatory effects that contains alkaloids such as tetrandrine (Tet), fangchinoline (Fan), cyclanoline (Cyc), and oblongine (Obl) as its main components. Various diluents and excipients could be included in SPRST preparation when necessary.

4. The specially processed extract of Radix Stephaniae tetrandrae (SPRST), a pharmaceutical compound-mixture displaying protective effect against ischaemic-reperfusion injury, features alkaloids as its main components including tetrandrine (Tet), fangchinoline (Fan), cyclanoline (Cyc), and oblongine (Obl). Various diluents and excipients could be included in SPRST when necessary.
